(19) United States
(12) Patent Application Publication
(10) Pub. No.: US 2008/0255353 A1
(43) Pub. Date: Oct. 16, 2008

(54) METHOD FOR PREPARING MELAMINE SALT OF BIS(PENTAERYTHRITOL PHOSPHATE) PHOSPHORIC ACID

(75) Inventors: Kuen-Yuan HWANG, Taipei (TW); An-Pang Tu, Taipei (TW); Gai-Chi Chen, Taipei (TW)

Correspondence Address: MORRISON & FOERSTER LLP, 755 PAGE MILL RD, PALO ALTO, CA, 94304-1018, US

(73) Assignee: Chang Chun Plastics Co., Ltd., Taipei (TW)

(21) Appl. No.: 12/020,454

(22) Filed: Jan. 25, 2008

(30) Foreign Application Priority Data
Apr. 13, 2007 (TW) 096112972

(51) Int. Cl. C07D 251/12

(52) U.S. Cl. 544/195

(57) ABSTRACT

A method for producing a melamine salt of bis(pentaerythritol phosphate) phosphoric acid is proposed. The method includes the steps of reacting and mixing pentaerythritol and diphosphorous pentaoxide in an extruder to carry out esterification to produce bis(pentaerythritol phosphate) phosphoric acid; and mixing bis(pentaerythritol phosphate) phosphoric acid with melamine or a derivative thereof to carry out quaternization in the presence of a solvent, followed by removing water to obtain a melamine salt of bis(pentaerythritol phosphate) phosphoric acid. Diphosphorous pentaoxide is used in the method as a reactant, so that no hydrogen chloride is produced and it is not necessary to recollect trichlorophosphoric acid. Moreover, in the method of the present invention, such that esterification time can be shortened and a high pressure in a closed reaction system that causes explosion can be avoided.

### CROSS-REFERENCE TO RELATED APPLICATIONS

This application claims the benefit of Taiwan application no. 096112972, filed Apr. 13, 2007, the disclosure of which is incorporated herein by reference in its entirety.

### BACKGROUND OF THE INVENTION

1. Field of the Invention

The present invention relates to methods for preparing a melamine salt of bis(pentaerythritol phosphate) phosphoric acid, and more particularly, to a method for preparing a melamine salt of bis(pentaerythritol phosphate) phosphoric acid by esterifying pentaerythritol and diphosphorous pentoxide.

2. Description of the Prior Art

The melamine salt of bis(pentaerythritol phosphate) phosphoric acid has a cyclic structure, which exhibits excellent heat stability during the thermal gravimetric analysis and the differential thermal analysis. The melamine salt of bis(pentaerythritol phosphate) phosphoric acid can be used in polyolefine, polyurethane, poly(phenylene oxide) (PPO), poly(methacrylate) (PMMA), polyester, and Poly(Butylene Terephthalate) (PBT) to decrease flammability, and thus the compound is used as a major component in intumescent flame retardants.

U.S. Pat. No. 4,154,930 discloses an amino-s-triazine salt of phosphoric acid as a flame retardant. U.S. Pat. No. 4,478,998 discloses a method for synthesizing a melamine salt of bis(pentaerythritol phosphate) phosphoric acid, by which the melamine salt is obtained by reacting melamine with oxytrichloride of bis(pentaerythritol phosphate) phosphoric acid. However, the method of the U.S. Pat. No. 4,478,998 that uses phosphorus oxychloride (POCl₃) as reactant, produces hydrogen chloride during reaction. When neutralizing hydrogen chloride, a large amount of sewage is produced. Further, because the viscosity of resin in the reaction is high, a high molar ratio of an excessive amount phosphorus oxychloride must be used for the reaction. Thus, the need to recollect phosphorus oxychloride liquid becomes another drawback in the method.

U.S. Pat. No. 6,737,526 discloses another method for mechanochemically synthesizing a melamine salt of bis(pentaerythritol phosphate) phosphoric acid by replacing phosphorous oxychloride and pentaerythritol with diphosphorous pentoxide, for grinding via a ball mill. However, diphosphorous pentoxide is highly reactive, and can easily react with water to form phosphoric acid, to react with other reactants to form metaphosphoric acid, or to react with ethers to form phosphoester. This poses numerous limitations on the selection of reaction solvents. Moreover, in the production process, organic solvents are used as media in a closed system, and water is produced during the reaction. This leads to an increased pressure in the closed system, thereby causing a safety issue. Moreover, the viscosity of pentaerythritol in the molten state is extremely high, which makes it difficult to agitate pentaerythritol. Therefore, a longer reaction time is required.

Accordingly, a method which can be used to produce a melamine salt of bis(pentaerythritol phosphate) phosphoric acid easily and safely is desired.

### SUMMARY OF INVENTION

It is therefore an objective of the present invention to provide a method for producing a melamine salt of bis(pentaerythritol phosphate) phosphoric acid, which does not produce hydrogen chloride.

It is another object of the present invention to provide a method for producing a melamine salt of bis(pentaerythritol phosphate) phosphoric acid, which can shorten the esterification time.

It is still another object of the present invention to provide a method for producing a melamine salt of bis(pentaerythritol phosphate) phosphoric acid, which can improve the the reaction system in safety aspect.

In accordance with the foregoing and other objectives, the present invention proposes a method for producing a melamine salt of bis(pentaerythritol phosphate) phosphoric acid. The method includes carrying out an esterification reaction in an extruder, of which pentaerythritol and diphosphorous pentaoxide are mixed to react with each other to form bis(pentaerythritol phosphate) phosphoric acid; and then carrying out a quaternization reaction by mixing and reacting bis(pentaerythritol phosphate) phosphoric acid, which is formed in the esterification reaction in the presence of a solvent, with melamine or a derivative thereof, followed by removing water to obtain a melamine salt of bis(pentaerythritol phosphate) phosphoric acid. In the method of the present invention, diphosphorous pentoxide is used as a reactant. The use of the method is free of generating hydrogen chloride as waste gas or creating the need to recollect trichlorophosphoric acid. Moreover, esterification can be carried out without the need to use organic solvents, such that the esterification time can be shortened and a high pressure in a closed reaction system that causes explosion can be avoided.

### DETAILED DESCRIPTION OF THE INVENTION

The method for producing a melamine salt of bis(pentaerythritol phosphate) phosphoric acid, according to the present invention, comprises the steps of (a) carrying out an esterification reaction by mixing and reacting pentaerythritol and diphosphorous pentaoxide to produce bis(pentaerythritol phosphate) phosphoric acid (b-PEPAP); and (b) carrying out a quaternization reaction by mixing bis(pentaerythritol phosphate) phosphoric acid obtained from esterification in step (a) with melamine or a derivative thereof, followed by removing water to obtain a melamine salt of bis(pentaerythritol phosphate) phosphoric acid (b-PEPAP MEL). The reaction mechanism is shown in the equation (I) below:

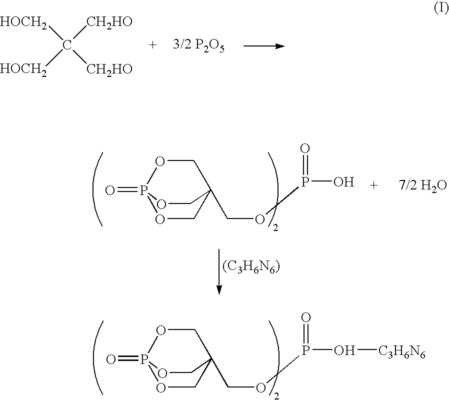

In the method according to the present invention, the esterification reaction of step (a) is carried out in an extruder. Types of the extruder can be classified into single-screw extruders and double-screw extruders. Extruders mainly exert external forces to mobilize materials to reach various degrees of mixing, kneading, shearing, and heating. In one embodiment, esterification in step (a) is carried out in a double-screw extruder, and the esterifying rate is determined in the outlet.

The esterification reaction is shown by the equation (II) below:

3/4 P₄O₁₀+2C₅H₁₂O₄→C₁₀H₁₇P₃O₁₂+7/2 H₂O (II)

Pentaerythritol and diphosphorous pentaoxide are fed into the double-screw extruder at predetermined proportions and feeding rate. In esterification reaction of step (a), the molar ratio of the reactants fed into the extruder has no significant effect on the production of bis(pentaerythritol phosphate) phosphoric acid. However, the feeding proportions of pentaerythritol and diphosphorous pentaoxide will affect the proportion of melamine salt produced, and change the property of the final product. If the proportion of pentaerythritol is inappropriate, carbon source will be insufficient. This will cause heat instability and poor flame retardation. Therefore, in step (a), pentaerythritol and diphosphorous pentaoxide are fed into the double-screw extruder at a molar ratio of 1:0.5 to 3 (preferably, at a molar ratio of 1:0.75 to 1.33), and more preferably, at a molar ratio of 1:1. The feeding rate varies with the screw rotation speed. If the feeding rate is too high, reactants are clogged in the inlet. If the feeding rate is too low, reactants are not thoroughly mixed and the retention time of the reactants in the feeding tube is prolonged. Therefore, the feeding rate is normally set at the range of 20 to 50 kg/hr.

In step (a), a preferred esterifying rate can be obtained by adjusting each of the operating parameters, such as the sleeve temperature and the screw rotation speed. The temperature is an important factor affecting the esterifying rate. That is, by increasing the sleeve temperature of the extruder, the esterifying rate is increased. However, when the temperature exceeds 200° C., the esterified product becomes carbonized. In the method according to the present invention, esterification is preferably performed at a temperature in the range of 30 to 200° C., and more preferably in the range of 50 to 140° C. Moreover, the screw rotation speed of the double-screw extruder also affects the esterifying rate. Low screw rotation speed increases the retention time of reactants in the screw, but decreases the shear stress. The decreased shear stress is unfavorable to an increase in the esterifying rate. Further, at an exceedingly high screw rotation speed, reactants are not retained in the screw long enough, thereby preventing the esterifying rate from increasing. In the method according to the present invention, esterification is performed preferably at the screw rotation speed of 10 to 300 min⁻¹, and more preferably of 50 to 200 min⁻¹. Generally, the preferred retention time for raw materials in the screw is between 0.5 to 4.5 minutes, more preferably between 1 to 4 minutes, and even more preferably between 1.5 to 3.5 minutes.

The quaternization reaction of step (b) of the present invention is carried out by mixing bis(pentaerythritol phosphate) phosphoric acid obtained from step (a) with melamine or a derivative thereof, such as benzoguanamine, in the presence of a solvent. In step (b), the amount of melamine or benzoguanamine added is preferably twice the number of moles of the pentaerythritol used. Water can be used as a solvent. Bis(pentaerythritol phosphate) phosphoric acid, melamine or benzoguanamine, and water are added into a reactor, such that quaternization can be carried out when the reacting temperature is increased from 30 to 90° C., and preferably from 70 to 90° C., while stirring. The reacting temperature is then lowered to remove water by filtration. Bis(pentaerythritol phosphate) melamine phosphate can accordingly be obtained after baking.

### EXAMPLE 1

Diphosphorous pentaoxide (P₂O₅) and pentaerythritol (PE) were fed into a double-screw extruder at a molar ratio of 1:1. Esterification was performed at a rotation speed of 100 min⁻¹ and a sleeve temperature in the range of 30 to 200° C. to obtain bis(pentaerythritol phosphate) phosphoric acid. Sampling was made at the outlet of the extruder, and a ³¹P-NMR map was used to calculate the esterifying rate. The result was recorded in Table 1.

An amount of 122.4 g of bis(pentaerythritol ester) phosphate, 111 g of melamine, and 500 g of pure water was added into a 1000 mL beaker, and the reacting temperature was increased to 90° C. while stirring. The duration of the process was 30 minutes. Then, the reacting temperature was decreased to 25° C. to remove water by filtration. A melamine salt of bis(pentaerythritol phosphate) phosphoric acid was obtained after baking.

### EXAMPLE 2-3

Steps in EXAMPLE 1 were repeated, and the screw rotation speed of the double-screw extruder was adjusted to carry out esterification according to Table 1. Sampling was made at the outlet of the extruder, and a ³¹P-NMR map was used to calculate the esterifying rate. The results were recorded in Table 1.

### EXAMPLE 4

Steps in EXAMPLE 1 were repeated, and the screw rotation speed of the double-screw extruder was adjusted to carry out esterification according to Table 1. Sampling was made at the outlet of the extruder, and a ³¹P-NMR map was used to calculate the esterifying rate. The result was recorded in Table 1.

### EXAMPLE 5-6

Steps in EXAMPLE 1 were repeated, and the feeding ratio of diphosphorous pentaoxide to pentaerythritol was adjusted to carry out esterification according to Table 1. Sampling was made at the outlet of the extruder, and a ³¹P-NMR map was used to calculate the esterifying rate. The results were recorded in Table 1.

### COMPARATIVE EXAMPLE 1

Steps in EXAMPLE 1 were repeated, and the sleeve temperature of the double-screw extruder was adjusted to carry out esterification according to Table 1. Sampling was made at the outlet of the extruder, and a ³¹P-NMR map was used to calculate the esterifying rate. The result was recorded in Table 1.

### COMPARATIVE EXAMPLE 2-3

Steps in EXAMPLE 1 were repeated, and the screw rotation speed of the double-screw extruder was adjusted to carry out esterification according to Table 1. Sampling was made at the outlet of the extruder, and a ³¹P-NMR map was used to calculate the esterifying rate. The results were recorded in Table 1.

TABLE 1

| | | | | | | | Comparative | Comparative | Comparative |
|---|---|---|---|---|---|---|---|---|---|
| | Example 1 | Example 2 | Example 3 | Example 4 | Example 5 | Example 6 | Example 1 | Example 2 | Example 3 |
| [P₂O₅]/[PE] | 1 | 1 | 1 | 1 | 1.33 | 0.75 | 1 | 1 | 1 |
| Feeding rate | | | | | | | | | |
| Screw rotation | 100 | 200 | 50 | 100 | 100 | 100 | 100 | 300 | 10 |
| speed | | | | | | | | | |
| (min⁻¹) | | | | | | | | | |
| Retention time | 1′31″ | 45″ | 3′06″ | 1′30″ | 1′33″ | 1′31″ | 1′31″ | 26″ | 5′12″ |
| of reactants in | | | | | | | | | |
| screw | | | | | | | | | |
| (min/sec) | | | | | | | | | |
| Sleeve | 30 | 30 | 30 | 50 | 50 | 50 | 50 | 30 | 30 |
| temperature | 40 | 40 | 40 | 90 | 90 | 90 | 90 | 40 | 40 |
| (° C.) | 50 | 50 | 50 | 100 | 100 | 100 | 100 | 50 | 50 |
| | 60 | 60 | 60 | 110 | 110 | 110 | 110 | 60 | 60 |
| | 90 | 90 | 90 | 120 | 120 | 120 | 120 | 90 | 90 |
| | 100 | 100 | 100 | 130 | 130 | 130 | 150 | 100 | 100 |
| | 110 | 110 | 110 | 130 | 130 | 130 | 160 | 110 | 110 |
| | 110 | 110 | 110 | 130 | 130 | 130 | 180 | 120 | 120 |
| | 120 | 120 | 120 | 130 | 130 | 130 | 190 | 130 | 130 |
| | 120 | 120 | 120 | 140 | 140 | 140 | 200 | 130 | 130 |
| Esterifying | 0.37 | 0.35 | 0.33 | 0.55 | 0.53 | 0.51 | Carbonization | 0.22 | 0.26 |
| rate | | | | | | | | | |

According to the results shown in Table 1, the sleeve temperature of the double-screw extruder was in the range of 30 to 200° C. and the screw rotation speed in the range of 10 to 300 min⁻¹ facilitated increases in the esterifying rate. The molar ratio of diphosphorous pentaoxide to pentaerythritol fed had no significant effect on the esterifying rate for producing bis(pentaerythritol phosphate) phosphoric acid.

What is claimed is:

1. A method for producing a melamine salt of bis(pentaerythritol phosphate) phosphoric acid, comprising the steps of:
(a) carrying out an esterification reaction by mixing and reacting pentaerythritol with diphosphorous pentaoxide in an extruder to obtain bis(pentaerythritol phosphate) phosphoric acid; and
(b) carrying out a quaternization reaction by mixing and reacting bis(pentaerythritol phosphate) phosphoric acid obtained from step (a) with melamine or a derivative thereof, in the presence of a solvent, followed by removing water to obtain a melamine salt of bis(pentaerythritol phosphate) phosphoric acid.

2. The method of claim 1, wherein a molar ratio of pentaerythritol to diphosphorous pentaoxide is 1:0.5 to 3.

3. The method of claim 2, wherein a molar ratio of pentaerythritol to diphosphorous pentaoxide is 1:0.75 to 1.33.

4. The method of claim 1, wherein the extruder is a double-screw extruder.

5. The method of claim 1, wherein a temperature of the extruder is ranged from 30 to 200° C.

6. The method of claim 5, wherein the temperature of the extruder is ranged from 50 to 140° C.

7. The method of claim 1, wherein a screw rotation speed of the extruder is ranged from 10 to 300 min⁻¹.

8. The method of claim 7, wherein the screw rotation speed of the extruder is ranged from 50 to 200 min⁻¹.

9. The method of claim 1, wherein the melamine derivative is benzoguanamine.

10. The method of claim 1, wherein a molar ratio of pentaerythritol to melamine or the derivative thereof is 1:2.
